(19) United States
(12) Plant Patent Application Publication
(10) Pub. No.: US 2008/0134398 P1
(43) Pub. Date: Jun. 5, 2008

(54) Geranium plant named 'Zonabriscal'

(75) Inventor: Theodorous Cornelis Maria van Kleinwee, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/633,833

(22) Filed: Dec. 5, 2006

(51) Int. Cl. A01H 5/00

(52) U.S. Cl. PLT/330

(57) ABSTRACT

A new Geranium plant particularly distinguished by the deep scarlet flower color, round plant structure with medium strong branching, green foliage with medium zone, and medium vigor.

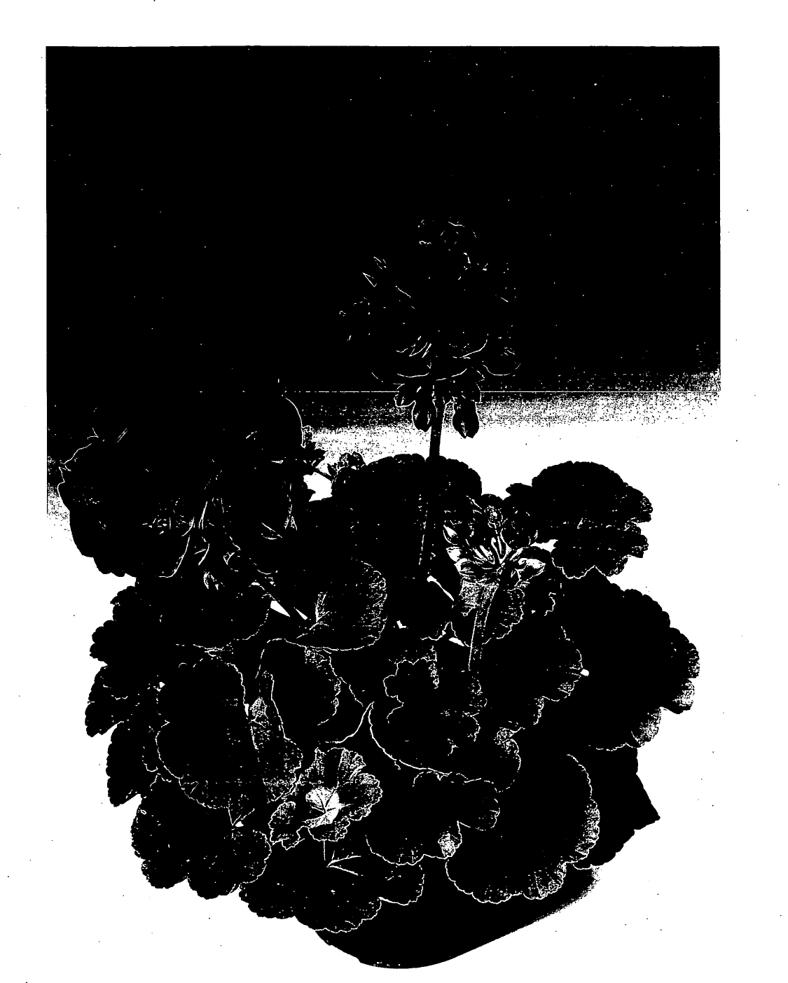

### LATIN NAME OF THE GENUS AND SPECIES OF THE PLANT CLAIMED

Pelargonium zonale (L.) L'Hérit. ex Ait

### VARIETAL DENOMINATION

‘Zonabriscal’

### BACKGROUND OF THE NEW PLANT

The present invention comprises a new Geranium, botanically known as Pelargonium zonale (L.) L'Hérit. ex Ait, and hereinafter referred to by the variety name ‘Zonabriscal.’

‘Zonabriscal’ is a product of a planned breeding program. The new cultivar ‘Zonabriscal’ has large, semi-double flowers, is relatively vigorous, but with a well-branched growth habit, and has good heat resistance.

‘Zonabriscal’ originates from a hybridization in a controlled breeding program in Enkhuizen, The Netherlands. The female parent was an unpatented hybrid seedling identified as no.1185 with deep scarlet red color and medium vigor with nice zone. The male parent of ‘Zonabriscal’ was an unpatented hybrid unknown seedling with unknown characteristics.

‘Zonabriscal’ was selected as one flowering plant within the progeny of the stated cross in 2001 in a controlled environment in Enkhuizen, The Netherlands.

The first act of asexual reproduction of ‘Zonabriscal’ was accomplished when vegetative cuttings were taken from the initial selection in the Fall of 2001 in a controlled environment in Enkhuizen, the Netherlands.

Horticultural examination of plants grown from cuttings of the plant initiated in April of 2002 in Enkhuizen, The Netherlands, and continuing thereafter, has demonstrated that the combination of characteristics as herein disclosed for ‘Zonabriscal’ are firmly fixed and are retained through successive generations of asexual reproduction.

‘Zonabriscal’ has not been observed under all possible environmental conditions. The phenotype may vary significantly with variations in environment such as temperature, light intensity and day length.

### BRIEF DESCRIPTION OF THE NEW VARIETY

The accompanying photographic drawing shows typical flower and foliage characteristics of ‘Zonabriscal’ with colors being as true as possible with an illustration of this type. The photographic drawing shows a flowering potted plant of the new variety.

### DETAILED BOTANICAL DESCRIPTION

The measurements were taken in Enkhuizen, The Netherlands, in August 2005 on plants that were growing in window boxes. Culture of these plants started mid February, 2005 in the greenhouse; plants were transferred to window boxes mid May, 2005.

### BRIEF SUMMARY OF INVENTION

The following observations, measurements, and comparisons describe plants grown outside in Enkhuizen, The Netherlands. The following traits have been repeatedly observed and are determined to be basic characteristics of the new variety. The combination of these characteristics distinguishes this Geranium as a new and distinct variety.

TABLE 1
DIFFERENCES BETWEEN THE NEW VARIETY ‘ZONABRISCAL’
AND A SIMILAR VARIETY

| | | ‘Americana Dark Red’(U.S. |
|---|---|---|
| | ‘Zonabriscal’ | Plant Patent No. 9,307) |
| Flower color | 44B | 46B |
| (upper side of upper petal) | | |
| Foliage size | Big | Medium |
| Petiole length | Long | Medium |

| | |
|---|---|
| PLANT | |
| Plant height (cm): | 30 |
| Plant height | 38 |
| (inflorescence included) (cm): | |
| Plant width (cm): | 65 |
| Plant shape: | Round |
| FOLIAGE | |
| Length (cm): | 7 |
| Width (cm): | 13 |
| Shape: | Reniform |
| Base shape: | Cordate |
| Base size (cm): | 5 |
| Apex shape: | Very weak lobes |
| Apex size (cm): | 4 |
| Color of upper surface: | 137B |
| Color of lower surface: | 146B |
| Color of zone: | 187A |
| Color of veins: | 146C |
| Margin: | Bicrenate |
| Petioles length (cm): | 6-11 |
| Diameter of petiole (mm): | 3 |
| Leaves smooth and hairy: | Only at nerves |
| Texture of leaves: | Upper surface smooth |
| Veins: | Palmate |
| STEM: | |
| Length of internodes (mm): | 10-45 |
| Color of stem: | 146C |
| Diameter (mm): | 9 |
| INFLORESCENCE | |
| Shape: | Round outline |
| Diameter (cm): | 12 |
| Umbel depth (cm): | 6 |
| Length of peduncle (cm): | 12-20 |
| Peduncle diameter (mm): | 3-4 |
| Color of peduncle: | 144A |
| Length of pedicel (cm): | 2.5-3.5 |
| Color of pedicel: | 183A |
| Number of flowers per | 30 |
| inflorescence: | |
| COROLLA | |
| Form: | Semi double |
| Shape: | Round outline, upper petals about the |
| | same size as the lower petals, |
| | cup shaped petals in two layers |
| Length of flower (cm): | 5.3 |
| Width of flower (cm): | 5 |
| Number of petals: | 5-9 |
| Length of petals | Upper 26 |
| (upper/lower) (mm): | |
| | Lower 29 |
| Width of petals | Upper 24 |
| (upper/lower) (mm): | |
| | Lower 29 |
| Shape petals: | Obovate, base acute, upper end is |
| | truncate or rounded, margin is entire |
| Color upper side of upper petals: | 44B |
| Color upper side of lower petals: | 44B |
| Color lower side of upper petals: | 43B |
| Color lower side of lower petals: | 43B |
| Number of petaloids: | 2-3; sometimes laciniated and crumbled |
| Color petaloids: | 44B |
| Fading color when flower | sometimes |
| starts aging: | |
| Duration of flowering: | Continuous flowering throughout the |
| | Summer |
| BUD (just before opening) | |
| Shape: | Narrow elliptic |
| Shape base of bud: | Truncate |
| Shape top of bud: | Acuminate |
| Sepals hairy and smooth: | Yes |
| Length (mm): | 10 |
| Width (mm): | 6 |
| Color: | 144A |
| Number of sepals: | 5 |
| Color of sepals: | 146C |
| Length of sepals (mm): | 10 |
| Width of sepals (mm): | 3 |
| REPRODUCTIVE ORGANS | |
| Number of lobes of stigma: | 5 |
| Color stigma: | 43A |
| Length of style (mm): | 6 |
| Color of style: | 43C |
| Number of anthers: | 8-10 |
| Length of anthers (mm): | 3 |
| Shape of anthers: | Ovate |
| Length filaments (mm): | 8 |
| Color of filaments: | 155D, Top 43B |
| Pollen present: | Yes |
| Color of pollen: | 32A |
| Fertility/seed set: | No |

Disease/pest resistance: No disease/pest resistance has been observed to date.

What is claimed is:

1. A new and distinct variety of Geranium plant, substantially as illustrated and described herein.
